(19) United States
(12) Patent Application Publication
(10) Pub. No.: US 2009/0157172 A1
(43) Pub. Date: Jun. 18, 2009

(54) STENTS WITH POLYMER-FREE COATINGS FOR DELIVERING A THERAPEUTIC AGENT

(75) Inventors: Jaydeep Y. Kokate, Minneapolis, MN (US); M. Arif Iftekhar, Santa Rosa, CA (US); Jan Weber, Maastricht (NL)

Correspondence Address: FISH & RICHARDSON PC, P.O. BOX 1022, MINNEAPOLIS, MN, 55440-1022, US

(73) Assignee: Boston Scientific Scrimed, Inc., Maple Grove, MN (US)

(21) Appl. No.: 12/176,450

(22) Filed: Jul. 21, 2008

Related U.S. Application Data
(60) Provisional application No. 60/951,551, filed on Jul. 24, 2007.

(51) Int. Cl. A61F 2/82

(52) U.S. Cl. 623/1.43; 623/1.42

(57) ABSTRACT

Described herein are implantable medical devices, such as intravascular stents, for delivering therapeutic agents to a patient, and methods for making such medical devices. The medical devices comprise a substrate having at least a cavity therein and a pellet disposed in the cavity. The pellet comprises a non-polymeric material having a plurality of pores therein. A therapeutic agent is disposed in at least some of the pores.

This application claims priority to U.S. Provisional Application No. 60/951,551 filed on Jul. 24, 2007, which is incorporated herein by reference in its entirety

### INTRODUCTION

Described herein are implantable medical devices, such as intravascular stents, for delivering therapeutic agents to a patient, and methods for making such medical devices. The medical devices comprise a substrate having at least a cavity therein and a pellet disposed in the cavity. The pellet comprises a non-polymeric material having a plurality of pores therein and a therapeutic agent disposed in at least some of the pores.

### 2.0 BACKGROUND

Medical devices have been used to deliver therapeutic agents locally to the body tissue of a patient. For example, stents having a coating containing a therapeutic agent, such as an anti-restenosis agent, have been used in treating or preventing restenosis. Currently, such medical device coatings include a therapeutic agent alone or a combination of a therapeutic agent and a polymer. Both of these types of coatings may have certain limitations.

Coatings containing a therapeutic agent without a polymer are generally ineffective in delivering the therapeutic agent since such coatings offer little or no control over the rate of release of the therapeutic agent. Specifically, the therapeutic agent is generally delivered in a burst release within a few hours. Therefore, many medical device coatings include a therapeutic agent and a polymer to provide sustained release of the therapeutic agent over time.

Though the use of polymers in coatings can provide control over the rate of release of the therapeutic agent therefrom, the use of such polymers in coatings may present certain other limitations. For example, the polymer in the coating may react adversely with the blood and cause thrombosis.

Moreover, some polymer coating compositions do not actually adhere to the surface of the medical device. In order to ensure that the coating compositions remain on the surface, the area of the medical device that is coated, such as a stent strut, is encapsulated with the coating composition. However, since the polymer does not adhere to the medical device, the coating composition is susceptible to deformation and damage during loading, deployment and implantation of the medical device. Any damage to the polymer coating may alter the therapeutic agent release profile and can lead to an undesirable increase or decrease in the therapeutic agent release rate.

Also, surfaces coated with compositions comprising a polymer may be subject to undesired adhesion to other surfaces. For instance, balloon expandable stents must be put in an unexpanded or “crimped” state before being delivered to a body lumen. During the crimping process coated stent struts are placed in contact with each other and can possibly adhere to each other. When the stent is expanded or uncrimped, the coating on the struts that have adhered to each other can be damaged, torn-off or otherwise removed. Moreover, if the polymer coating is applied to the inner surface of the stent, it may stick or adhere to the balloon used to expand the stent when the balloon contacts the inner surface of the stent during expansion. Such adherence to the balloon may prevent a successful deployment of the medical device.

Similar to balloon-expandable stents, polymer coatings on self-expanding stents can also interfere with the delivery of the stent. Self-expanding stents are usually delivered using a pull-back sheath system. When the system is activated to deliver the stent, the sheath is pulled back, exposing the stent and allowing the stent to expand itself. As the sheath is pulled back it slides over the outer surface of the stent. Polymer coatings located on the outer or abluminal surface of the stent can adhere to the sheath as it is being pulled back and disrupt the delivery of the stent.

Accordingly, there is a need for medical devices that have little or no polymer and that can release an effective amount of a therapeutic agent in a controlled release manner while avoiding the disadvantages of current coatings for medical devices that include a polymer. Additionally, there is a need for methods of making such medical devices.

### 3.0 SUMMARY

These and other objectives are addressed by the embodiments described herein. The embodiments described herein include medical devices that are capable of releasing a therapeutic agent in a controlled release manner as well as methods for making such devices.

In one embodiment, the medical device, which can be an implantable stent, comprises a stent sidewall structure having a surface and at least one cavity, having first and second opposing ends, disposed within the stent sidewall structure. The first end of the cavity comprises an opening that is in fluid communication with the stent sidewall structure surface and the second end of the cavity comprises the bottom of the cavity. Also, at least one pellet is disposed within the cavity that comprises a non-polymeric material having a plurality of pores therein. A therapeutic agent is disposed in at least some of the pores of the pellet. The stent sidewall structure surface can be free of any coating. In some embodiments, the pellet has first and second opposing ends, in which the first end of the pellet faces toward the first end of the cavity and the second end of the pellet faces toward the second end of the cavity.

Furthermore, at least some of the pores of the pellets can have different pore sizes. In some instances, the pores are arranged in a manner to form a pore size gradient in the pellet. The pore size gradient can extend from the first end of the pellet to the second end of the pellet. Also, the pores having the largest pore size can be disposed proximate the first end of the pellet.

Moreover, in some embodiments, the pellet can comprise one or more layers. For example, the pellet can include a first layer comprising pores having a first pore size and a second layer comprising pores having a second pore size that is different from the first pore size. Also, the layers can be arranged in a manner to form a pore size gradient in the pellet. In some instances, the pores having the largest pore size are disposed in the layer proximate the first end of the pellet.

In another embodiment, the medical device can be an implantable intravascular stent comprising a stent sidewall structure comprising a plurality of struts each having an abluminal surface and a luminal surface. There is at least one cavity, having first and second opposing ends, disposed within a strut wherein the first end of the cavity comprises an opening that is in fluid communication with the abluminal surface of the strut and the second end of the cavity comprises the bottom of the cavity. At least one pellet comprising a non-polymeric material, having a plurality of pores therein, is disposed in the cavity. The pellet has first and second opposing ends, and the first end of the pellet faces toward the first end of the cavity and the second end of the pellet faces toward the second end of the cavity. Also, at least some of the pores have different pore sizes and the pores are arranged in a manner to form a pore size gradient in the pellet, in which the pores having the largest pore size are disposed proximate the first end of the pellet. An anti-restenosis agent is disposed within at least some of the pores of the pellet.

In yet another embodiment, the medical device can be an implantable intravascular stent comprising a stent sidewall structure comprising a plurality of struts each having an abluminal surface and a luminal surface. There is at least one cavity, having first and second opposing ends, disposed within a strut. The first end of the cavity comprises an opening that is in fluid communication with the abluminal surface of the strut and the second end of the cavity comprises the bottom of the cavity. Also, there is at least one pellet comprising a non-polymeric material, having a plurality of pores therein, disposed in the cavity. The pellet has first and second opposing ends, and the first end of the pellet faces toward the first end of the cavity and the second end of the pellet faces toward the second end of the cavity. In addition, the pellet comprises a first layer comprising pores having a first pore size, a second layer comprising pores having a second pore size that is smaller than the first pore size, and a third layer comprising pores having a third pore size that is smaller than the second pore size. The first, second and third layers are arranged in a manner to form a pore size gradient in the pellet, in which the first layer is disposed proximate the first end of the pellet. An anti-restenosis agent is disposed within at least some of the pores of the pellet.

Also described herein are methods for making the medical device. In one embodiment, the method for making the medical device, which can be a stent, comprises providing a stent having a stent sidewall structure having a surface and at least one cavity, having first and second opposing ends, disposed within the stent sidewall structure. The first end of the cavity comprises an opening that is in fluid communication with the stent sidewall structure surface and the second end of the cavity comprises the bottom of the cavity. The method further comprises disposing at least one pellet into the cavity. The pellet comprises a non-polymeric material having a plurality of pores therein; as well as first and second opposing ends. The first end of the pellet faces toward the first end of the cavity and the second end of the pellet faces toward the second end of the cavity. At least some of the pores have different pore sizes and the pores are arranged in a manner to form a pore size gradient in the pellet. The method also comprises disposing a therapeutic agent in at least some of the pores of the pellet.

In another embodiment, the method for making the medical device, such as an implantable stent, comprises providing a stent having a stent sidewall structure having a surface and at least one cavity, having first and second opposing ends, disposed within the stent sidewall structure. The first end of the cavity comprises an opening that is in fluid communication with the stent sidewall structure surface and the second end of the cavity comprises the bottom of the cavity. The method further comprises forming a pellet in the cavity, wherein the pellet has a plurality of layers, and first and second opposing ends. The first end of the pellet faces toward the first end of the cavity and the second end of the pellet faces toward the second end of the cavity. The step of forming the pellet comprises disposing a first solid, non-polymeric material into the cavity to form a first layer of the pellet, wherein the first layer has a plurality of pores having a first pore size. A second solid, non-polymeric material is disposed into the cavity to form a second layer of the pellet disposed over the first layer, wherein the second layer has a plurality of pores having a second pore size. The method further comprises disposing a therapeutic agent in at least some of the pores of the first and second layers.

### 4.0 BRIEF DESCRIPTION OF THE DRAWINGS

Certain embodiments will be explained with reference to the following drawings.

FIG. 1 shows a cross-sectional view of an example of a medical device substrate having cavities therein and pellets, having a plurality of pores, disposed in the cavities.

FIG. 2 shows a cross-sectional view of another example of a medical device substrate having cavities therein and pellets, having a plurality of pores, disposed in the cavities, in which a pore size gradient is present in the pellets.

FIG. 3 shows a cross-sectional view of another example of a medical device substrate having cavities therein and pellets, having a plurality of pores and layers, disposed in the cavities, in which a pore size gradient is present in the pellets.

FIG. 4A shows a cross-sectional view of another embodiment of the medical device comprising cavities and pellets disposed in the cavities.

FIG. 4B shows a cross-sectional view of yet another embodiment of the medical device comprising cavities and pellets disposed in the cavities.

FIG. 5 shows a peripheral view of an embodiment of an intravascular stent.

FIGS. 6A-6C show a method of preparing a medical device comprising cavities and pellets disposed in the cavities.

FIGS. 7A-7C show another method of preparing a medical device comprising cavities and pellets disposed in the cavities.

### 5.0 DETAILED DESCRIPTION

### 5.1 The Medical Device

The medical devices described herein generally include a substrate having at least one surface. For instance, in the case where the medical device is an intravascular stent, the substrate is the stent sidewall structure and the surface is the abluminal surface of the stent. A cavity is disposed in the substrate and a pellet comprising a non-polymeric material having a plurality of pores therein is disposed in the cavity. A therapeutic agent is disposed in at least some of the pores for delivery to a patient.

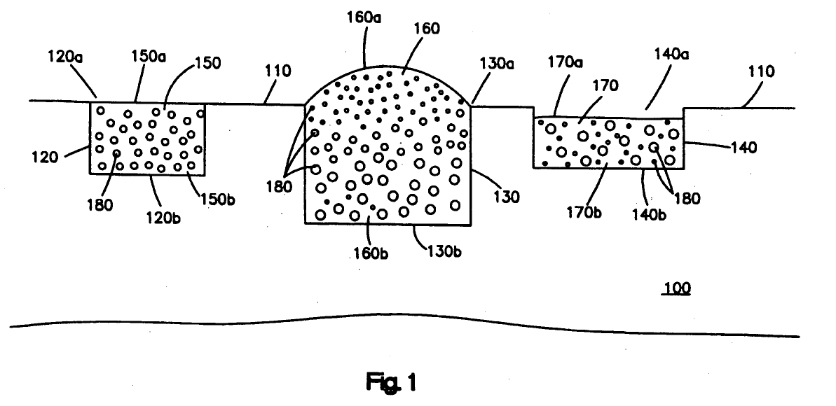

FIG. 1 shows one embodiment of the medical device, which can be a stent. The medical device comprises a substrate 100 having a surface 110. In this embodiment, the surface 110 is free of any coating, i.e., is not covered by a coating. In other embodiments, a coating may be disposed on at least a portion of the surface 110. As shown in the figure, there are three cavities 120, 130 and 140 disposed in the substrate 100. Each cavity comprises two opposing ends 120a and 120b, 130a and 130b, and 140a and 140b. Each first end 120a, 130a and 140a comprises an opening that is in fluid communication with the surface 110. Each second end 120b, 130b and 140b comprises the bottom of the cavity. In this embodiment, since the cavity has a bottom, the cavity does not extend through the entire substrate. In other embodiments the cavity can extend through the entire substrate. For instance, if the cavity is disposed in a stent strut the cavity can extend through the strut. Also, as shown in FIG. 1, the cavities in a single substrate can have different sizes or geometries or cross-sectional shapes.

Pellets 150, 160 and 170 are disposed in each of the cavities 120, 130 and 140. The pellets comprise a non-polymeric material. In some embodiments, the pellet is substantially free of any polymer, i.e. no polymer is intentionally included. Also, the pellets, 150, 160 and 170 each comprise two opposing ends 150a and 150b, 160a and 160b, and 170a and 170b. Each first end 150a, 160a and 170a of the pellets 150, 160 and 170 faces toward the opening of a cavity. Each second end 150b, 160b and 170b of the pellets 150, 160 and 170 faces toward the bottom of a cavity.

In certain embodiments, the pellet does not extend beyond the opening of the cavity in which the pellet is disposed. In FIG. 1, pellets 150 and 170 are examples of such pellets. Pellet 150 extends up to the opening 120a and pellet 170 does not extend beyond the opening 140a. In other embodiments, the pellet does extend beyond the opening of the cavity in which it is disposed. Pellet 160 is an example of such a pellet where the first end 160a of the pellet 160 extends past the opening 130a of the cavity 130.

Furthermore, as shown in FIG. 1, the pellets are comprised of a material having a plurality of pores 180 therein. A therapeutic agent (not shown) is disposed in at least some of the pores 180. The pores of the pellet can be of a generally uniform pore size such as the pores of pellet 150. Alternatively, the pores can have varying pore sizes throughout the pellet as shown in pellets 160 and 170. In pellet 160, the pores 180 are arranged in a manner such that a pore size gradient is formed. The pores 180 at the second end 160b of the pellet 160 are the largest and the pores 180 at the first end 160a of the pellet 160 are the smallest, while the pores in the middle of the pellet have pore sizes that lie between the smallest and largest sizes. In pellet 170, pores 180 having small and large sizes are dispersed among each other.

Porosity and surface area of porous pellets can be measured by various techniques such as, but not limited to, physical gas absorption, helium pycnometry and mercury porosimetry. Physical gas absorption uses inert gas such as argon, nitrogen, krypton or carbon dioxide to determine surface area or total pore volume of the porous material. Helium pycnometry is a technique used to obtain information on the true density of solids using helium, which can enter even the smallest voids or pores. Mercury porosimetry uses the non-wetting properties of mercury to gain information of the porous characteristics of solid materials.

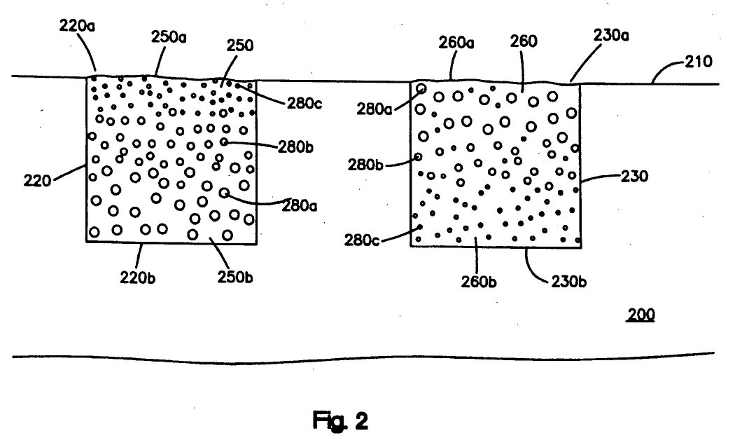

FIG. 2 shows another embodiment of a medical device having a substrate 200, a surface 210 and cavities 220, 230 disposed in the substrate. Each cavity comprises two opposing ends 220a and 220b, and 230a and 230b. Each first end 220a and 230a comprises an opening that is in fluid communication with the surface 210. Each second end 220b and 230b of the cavities 220 and 230 comprises the bottom of the cavity.

Pellets 250 and 260 each comprise two opposing ends 250a and 250b, and 260a and 260b. Each first end 250a and 260a faces toward an opening of a cavity. Each second end 250b and 260b of the pellets 250, 260 faces toward the bottom of a cavity. Similar to the pellets shown in FIG. 1, the pellets are comprised of a material having a plurality of pores 280a, 280b and 280c therein. A therapeutic agent (not shown) is disposed in at least some of the pores. In this embodiment, the pores are arranged in a manner such that a pore size gradient is formed. In pellet 250, the pores 280a that are proximate the second end 250b of the pellet 250 are generally the largest in pore size and the pores 280c that are proximate first end 250a of the pellet 250 are generally the smallest in pore size. The pores 280b in the middle of the pellet 250 have pore sizes that are generally between the smallest and largest pore sizes. In pellet 260, the pores 280a that are proximate the first end 260a of the pellet 260 are generally the largest in pore size and the pores 280c that are proximate the second end 260b of the pellet 260 are generally the smallest in pore size. The pores 280b in the middle of the pellet 260 generally have pore sizes that are between the smallest and largest pore sizes. The advantages of having a pore size gradient include creating a variety of drug release profiles

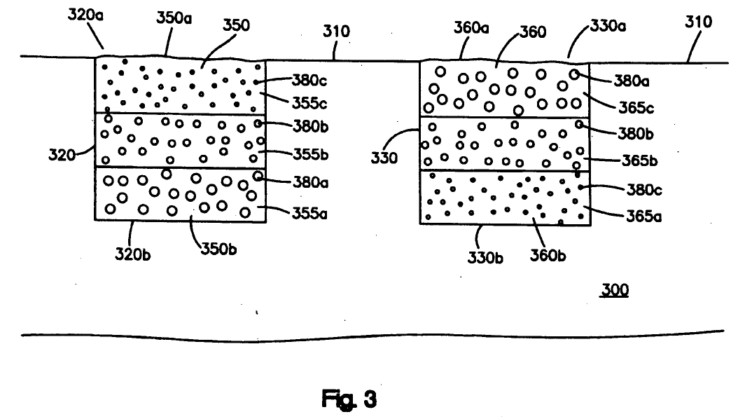

FIG. 3 shows another embodiment of a medical device having a substrate 300, a surface 310 and cavities 320 and 330 disposed in the substrate. Each cavity comprises two opposing ends 320a and 320b, and 330a and 330b. Each first end 320a and 330a comprises an opening that is in fluid communication with the surface 310. Each second end 320b and 330b of the cavities 320 and 330 comprises the bottom of the cavity.

Like the pellets described above, pellets 350 and 360 each comprise two opposing ends 350a and 350b, and 360a and 360b. Each first end 350a and 360a faces toward an opening of a cavity. Each second end 350b and 360b of the pellets 350 and 360 faces toward the bottom of a cavity. The pellets 350, 360 are comprised of layers 355a, 355b and 355c, and 365a, 365b and 365c of materials having a plurality of pores 380a, 380b and 380c therein. A therapeutic agent (not shown) is disposed in at least some of the pores. The layers can have various thicknesses.

In this embodiment, each layer of a pellet has pores of different pore sizes. For example, with respect to pellet 350, the first layer 355a has pores 380a that have a first pore size, i.e. the pores predominantly have this pore size but there may be some pores having different pore sizes. The second layer 355b, which is disposed on the first layer 355a, has pores 380b having a second pore size that is smaller than the first pore size. The third layer 355c of pellet 350, which is disposed on the second layer 355b, has pores 380c having a third pore size that is smaller than the second pore size. In this pellet 350, the layers 355a, 355b and 355c are arranged in a manner to form a pore size gradient in the pellet, which in this case extends from the first end 350a of the pellet to the second end 350b. Also, in this pellet 350, the pores 380c having the smallest pore size are disposed in the layer proximate the first end of the pellet 350a.

The other pellet 360 of the medical device shown in FIG. 3 also comprises three layers 365a, 365b, and 365c. Each layer of this pellet 360 also has pores of different pore sizes. The first layer 365a has pores 380c having a first pore size. The second layer 365b, which is disposed on the first layer 365a, has pores 380b having a second pore size that is larger than the first pore size. The third layer 365c of pellet 360, which is disposed on the second layer 365b, has pores 380a having a third pore size that is larger than the second pore size. In this pellet 360, the layers 365a, 365b and 365c are also arranged in a manner to form a pore size gradient in the pellet, which in this case extends from the first end 360a of the pellet to the second end 360b. Moreover, in this pellet 360, the pores 380a having the largest pore size are disposed in the layer proximate the first end of the pellet 360a.

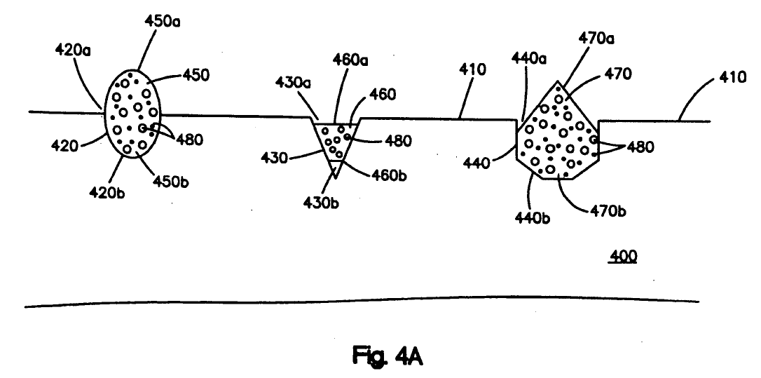

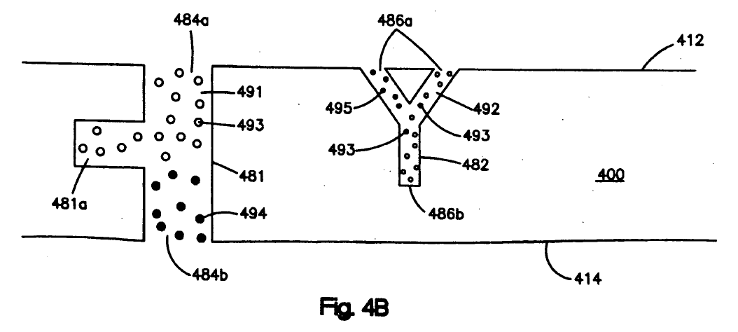

FIG. 4A shows a medical device having a substrate 400, a surface 410 and three cavities 420, 430 and 440 disposed in the substrate 400. Each cavity comprises two opposing ends 420a and 420b, 430a and 430b, and 440a and 440b. Each first end 420a, 430a and 440a comprises an opening that is in fluid communication with the surface 410. Each second end 420b, 430b and 440b of the cavities 420, 430 and 440 comprises a bottom of a cavity. The three cavities have different geometries with different cross-sectional shapes. Specifically, cavity 420 has a U-shaped cross-section, cavity 430 has a V-shaped or triangular cross-section and cavity 440 has a modified-U-shaped cross-section. In other embodiments, such as those shown in FIG. 4B, the cavities can have other geometries and cross-sections.

Also, as shown in FIG. 4A, the pellets, which have a plurality of pores 480, disposed in the cavities do not necessarily have to conform to the geometry or shape of the cavities, or be confined within the cavity. For instance, pellet 450, which has first and second ends 450a, 450b, conforms to the cavity but then extends beyond the opening 420a of the cavity 420. Pellet 460, which has first and second ends 460a, 460b, is contained in cavity 430 but does not fill or completely conform to cavity 430. Pellet 470, which has first and second ends 470a, 470b, extends beyond opening 440a of the cavity 440 but does not completely conform to the entire cavity 440.

FIG. 4B shows a medical device, such as a stent strut, having a substrate 400, with an abluminal surface 412 and a luminal surface 414. The abluminal surface is the surface of the medical device that faces away from a body lumen and the luminal surface is the surface of the medical device that faces towards a body lumen. As shown in FIG. 4B, the medical device substrate 400 has two cavities 481 and 482 disposed in the substrate 400. Each cavity comprises two opposing ends 484a and 484b and 486a and 486b. The first end 484a of cavity 481 comprises an opening that is in fluid communication with the abluminal surface 412. The second end 484b comprises an opening that is in fluid communication with luminal surface 414. Also, cavity 481 has a portion 481a that gives the cavity a T-shaped cross-section. With respect to cavity 482, the first end 486a comprises two openings that are in fluid communication with the abluminal surface 412 and the second end 486b of cavity 481 comprises the bottom of the cavity. As shown in FIG. 4B, cavity 482 has a Y-shaped cross-section.

As shown in FIG. 4B, pellets 491 and 492, which have a plurality of pores 493, are disposed in the cavities. The shapes of the pellets and cavities prevent the pellets from easily being removed from the cavities. Moreover, if the pellets shrink or otherwise change shape such pellet and cavity shapes will prevent the pellets from unintentionally falling out of the cavity.

Additionally, as shown in the embodiment of FIG. 4B, pellets 491 and 492 each include a different therapeutic agent. As shown in FIG. 4B, pellet 491 includes therapeutic agent 494 disposed in at least some of the pores 493 and pellet 492 includes therapeutic agent 495 disposed in at least some of the pores 493. However, in other embodiments described herein, all pellets disposed in cavities of a medical substrate can include the same therapeutic agent.

### 5.1.1 Types of Medical Devices

The medical devices described herein can be implanted or inserted into the body of a patient. Suitable medical devices include, but are not limited to, stents, surgical staples, catheters, such as balloon catheters, central venous catheters, and arterial catheters, guide wires, cannulas, cardiac pacemaker leads or lead tips, cardiac defibrillator leads or lead tips, implantable vascular access ports, blood storage bags, blood tubing, vascular or other grafts, intra aortic balloon pumps, heart valves, cardiovascular sutures, total artificial hearts and ventricular assist pumps, and extra corporeal devices such as blood oxygenators, blood filters, septal defect devices, hemodialysis units, hemoperfusion units and plasmapheresis units.

Suitable medical devices include, but are not limited to, those that have a tubular or cylindrical like portion. For example, the tubular portion of the medical device need not be completely cylindrical. The cross-section of the tubular portion can be any shape, such as rectangle, a triangle, etc., not just a circle. Such devices include, but are not limited to, stents, balloon catheters, and grafts. A bifurcated stent is also included among the medical devices which can be fabricated by the methods described herein.

In addition, the tubular portion of the medical device may be a sidewall that may comprise a plurality of struts defining a plurality of openings. The sidewall defines a lumen. The struts may be arranged in any suitable configuration. Also, the struts do not all have to have the same shape or geometric configuration. When the medical device is a stent comprising a plurality of struts, the surface is located on the struts. Each individual strut has an outer surface adapted for exposure to the body tissue of the patient, an inner surface, and at least one side surface between the outer surface and the inner surface.

Medical devices that are particularly suitable for the embodiments described herein include any kind of stent for medical purposes which is known to the skilled artisan. Preferably, the stents are intravascular stents that are designed for permanent implantation in a blood vessel of a patient. In certain embodiments, the stent comprises an open lattice sidewall stent structure. In preferred embodiments, the stent is a coronary stent. Other suitable stents include, for example, vascular stents such as self-expanding stents and balloon expandable stents. Examples of self-expanding stents useful in the embodiments described herein are illustrated in U.S. Pat. Nos. 4,655,771 and 4,954,126 issued to Wallsten and U.S. Pat. No. 5,061,275 issued to Wallsten et al. Examples of appropriate balloon-expandable stents are shown in U.S. Pat. No. 5,449,373 issued to Pinchasik et al.

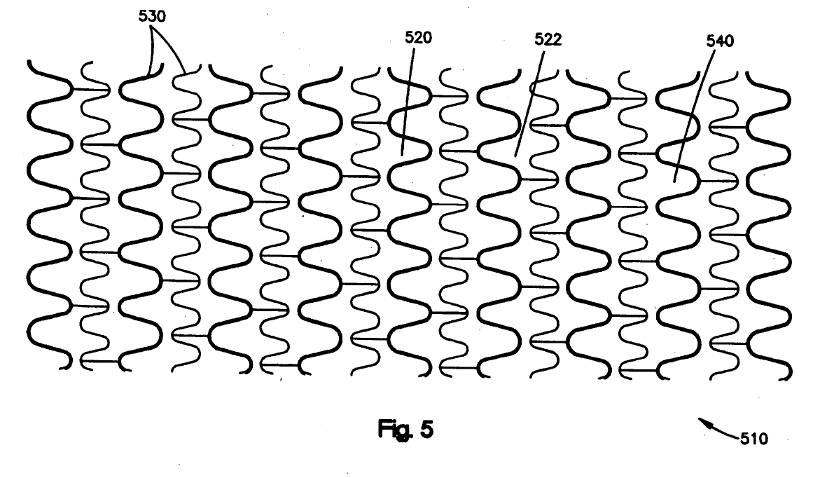

FIG. 5 shows an example of a medical device that is suitable for use in the embodiments described herein. This figure shows a peripheral view of an implantable intravascular stent 510. As shown in FIG. 5, the intravascular stent 510 is generally cylindrical in shape. Stent 510 includes a sidewall 520 which comprises a plurality of struts 530 and at least one opening 540 in the sidewall 520. Generally, the opening 540 is disposed between adjacent struts 530. Also, the sidewall 520 may have a first sidewall surface 522 and an opposing second sidewall surface, which is not shown in FIG. 5. The first sidewall surface 522 can be an outer or abluminal sidewall surface, which faces a body lumen wall when the stent is implanted, or an inner or luminal sidewall surface, which faces away from the body lumen surface. Likewise, the second sidewall surface can be an abluminal sidewall surface or a luminal sidewall surface. In certain embodiments, at least one strut comprises an abluminal surface, which forms part of the abluminal surface of the stent, and at least one strut comprises a luminal surface opposite the abluminal surface of the strut, which forms part of the luminal surface of the stent.

In some embodiments, the abluminal surface of the stent sidewall structure comprises at least one cavity and the luminal surface is free of cavities. In other embodiments, the cavity or cavities can be located on a low-stress bearing part of the stent sidewall structure.

When the coatings described herein are applied to a stent having openings in the stent sidewall structure, in certain embodiments, it is preferable that the coatings conform to the surface of the stent so that the openings in the sidewall stent structure are preserved, e.g. the openings are not entirely or partially occluded with coating material.

The framework of suitable stents may be formed through various methods known in the art. The framework may be welded, molded, laser cut, electro-formed, or consist of filaments or fibers which are wound or braided together in order to form a continuous structure.

Suitable substrates of the medical device (e.g. stents) described herein may be fabricated from a metallic material, ceramic material, polymeric or non-polymeric material, or a combination thereof (see Sections 5.1.1.1 to 5.1.1.3 infra.). Preferably, the materials are biocompatible. The material may be porous or non-porous, and the porous structural elements can be microporous or nanoporous.

### 5.1.1.1. Metallic Materials for Medical Devices

In certain embodiments, the medical devices described herein can comprise a substrate which is metallic. Suitable metallic materials useful for making the substrate include, but are not limited to, metals and alloys based on titanium (such as nitinol, nickel titanium alloys, thermo memory alloy materials), stainless steel, gold, iron, magnesium, platinum, iridium, molybdenum, niobium, palladium, chromium, tantalum, nickel chrome, or certain cobalt alloys including cobalt chromium nickel alloys such as Elgiloy® and Phynox®, or a combination thereof. Other metallic materials that can be used to make the medical device include clad composite filaments, such as those disclosed in WO 94/16646.

In some embodiments, the metal is a radiopaque material that makes the medical device visible under X-ray or fluoroscopy. Suitable materials that are radiopaque include, but are not limited to, gold, tantalum, platinum, bismuth, iridium, zirconium, iodine, titanium, barium, silver, tin, alloys of these metals, or a combination thereof.

Furthermore, although the embodiments described herein can be practiced by using a single type of metal to form the substrate, various combinations of metals can also be employed. The appropriate mixture of metals can be coordinated to produce desired effects when incorporated into a substrate.

### 5.1.1.2. Ceramic Materials for Medical Devices

In certain embodiments, the medical devices described herein can comprise a substrate which is ceramic. Suitable ceramic materials used for making the substrate include, but are not limited to, oxides, carbides, or nitrides of transition elements such as titanium oxides, hafnium oxides, iridium oxides, chromium oxides, aluminum oxides, zirconium oxides, transition metal oxides, platinum oxides, tantalum oxides, niobium oxides, tungsten oxides, rhodium oxides, or a combination thereof. Silicon based materials, such as silica, may also be used. Furthermore, although certain embodiments described herein can be practiced by using a single type of ceramic to form the substrate, various combinations of ceramics can also be employed. The appropriate mixture of ceramics can be coordinated to produce desired effects when incorporated into a substrate.

### 5.1.1.3. Polymeric Materials for Medical Devices

In certain embodiments, the medical devices described herein can comprise a substrate which is polymeric. In other embodiments, the material can be a non-polymeric material. The polymer(s) useful for forming the components of the medical devices should be ones that are biocompatible and avoid irritation to body tissue. The polymers can be biostable or bioabsorbable. Suitable polymeric materials useful for making the substrate include, but are not limited to, isobutylene-based polymers, polystyrene-based polymers, polyacrylates, and polyacrylate derivatives, vinyl acetate-based polymers and its copolymers, polyurethane and its copolymers, silicone and its copolymers, ethylene vinyl-acetate, polyethylene terephtalate, thermoplastic elastomers, polyvinyl chloride, polyolefins, cellulosics, polyamides, polyesters, polysulfones, polytetrafluorethylenes, polycarbonates, acrylonitrile butadiene styrene copolymers, acrylics, polylactic acid, polyglycolic acid, polycaprolactone, polylactic acid-polyethylene oxide copolymers, cellulose, collagens, chitins, or a combination thereof.

Other polymers that are useful as materials for making the substrate include, but are not limited to, dacron polyester, poly(ethylene terephthalate), polycarbonate, polymethylmethacrylate, polypropylene, polyalkylene oxalates, polyvinylchloride, polysiloxanes, nylons, poly(dimethyl siloxane), polycyanoacrylates, polyphosphazenes, poly(amino acids), ethylene glycol I dimethacrylate, poly(methyl methacrylate), poly(2-hydroxyethyl methacrylate), polytetrafluoroethylene poly(HEMA), polyhydroxyalkanoates, poly(glycolide-lactide) co-polymer, poly(β-hydroxybutyrate), polydioxanone, poly(γ-ethyl glutamate), polyiminocarbonates, poly(ortho ester), polyanhydrides, styrene isobutylene styrene, polyetheroxides, polyvinyl alcohol, poly-2-hydroxy-butyrate, polycaprolactone, poly(lactic-co-clycolic)acid, Teflon, alginate, dextran, cotton, derivatized versions thereof, (i.e., polymers which have been modified to include, for example, attachment sites or cross-linking groups, e.g., arginine-glycine-aspartic acid RGD, in which the polymers retain their structural integrity while allowing for attachment of cells and molecules, such as proteins and/or nucleic acids), or a combination thereof.

The polymers may be dried to increase their mechanical strength. The polymers may then be used as the base material to form a whole or part of the substrate.

Furthermore, although the embodiments described herein can be practiced by using a single type of polymer to form the substrate, various combinations of polymers can also be employed. The appropriate mixture of polymers can be coordinated to produce desired effects when incorporated into a substrate.

### 5.1.2 Non-Polymeric Materials For Making The Pellets

The non-polymeric materials that can be used to form the pellets include without limitation the metal and metal oxides described above that can be used to make the medical devices. Preferred metals and metal oxides that can be used to form the pellets include, without limitation, titanium dioxide, in anatase or rutile form; silica; hydroxyl-apatite; stainless steel or gold.

### 5.1.3 Therapeutic Agents

The phrase “therapeutic agent” as used herein encompasses drugs, genetic materials, and biological materials and can be used interchangeably with “biologically active material”. The term “genetic materials” means DNA or RNA, including, without limitation, DNA/RNA encoding a useful protein stated below, intended to be inserted into a human body including viral vectors and non-viral vectors.

The term “biological materials” include cells, yeasts, bacteria, proteins, peptides, cytokines and hormones. Examples for peptides and proteins include vascular endothelial growth factor (VEGF), transforming growth factor (TGF), fibroblast growth factor (FGF), epidermal growth factor (EGF), cartilage growth factor (CGF), nerve growth factor (NGF), keratinocyte growth factor (KGF), skeletal growth factor (SGF), osteoblast-derived growth factor (BDGF), hepatocyte growth factor (HGF), insulin-like growth factor (IGF), cytokine growth factors (CGF), platelet-derived growth factor (PDGF), hypoxia inducible factor-1 (HIF-1), stem cell derived factor (SDF), stem cell factor (SCF), endothelial cell growth supplement (ECGS), granulocyte macrophage colony stimulating factor (GM-CSF), growth differentiation factor (GDF), integrin modulating factor (IMF), calmodulin (CaM), thymidine kinase (TK), tumor necrosis factor (TNF), growth hormone (GH), bone morphogenic protein (BMP) (e.g., BMP-2, BMP-3, BMP-4, BMP-5, BMP-6 (Vgr-1), BMP-7 (PO-1), BMP-8, BMP-9, BMP-110, BMP-11, BMP-12, BMP-14, BMP-15, BMP-16, etc.), matrix metalloproteinase (MMP), tissue inhibitor of matrix metalloproteinase (TIMP), cytokines, interleukin (e.g. IL-1, IL-2, IL-3, IL-4, IL-5, IL-6, IL-7, IL-8, IL-9, IL-10, IL-11, IL-12, IL-15, etc.), lymphokines, interferon, integrin, collagen (all types), elastin, fibrillins, fibronectin, vitronectin, laminin, glycosaminoglycans, proteoglycans, transferrin, cytotactin, cell binding domains (e.g., RGD), and tenascin. Currently preferred BMP's are BMP-2, BMP-3, BMP-4, BMP-5, BMP-6, BMP-7. These dimeric proteins can be provided as homodimers, heterodimers, or combinations thereof, alone or together with other molecules. Cells can be of human origin (autologous or allogeneic) or from an animal source (xenogeneic), genetically engineered, if desired, to deliver proteins of interest at the transplant site. The delivery media can be formulated as needed to maintain cell function and viability. Cells include progenitor cells (e.g., endothelial progenitor cells), stem cells (e.g., mesenchymal, hematopoietic, neuronal), stromal cells, parenchymal cells, undifferentiated cells, fibroblasts, macrophage, and satellite cells.

Other suitable therapeutic agents include:
anti-thrombogenic agents such as heparin, heparin derivatives, urokinase, and PPack (dextrophenylalanine proline arginine chloromethylketone);
anti-proliferative agents such as enoxaprin, angiopeptin, or monoclonal antibodies capable of blocking smooth muscle cell proliferation, hirudin, acetylsalicylic acid, tacrolimus, everolimus, pimecrolimus, sirolimus, zotarolimus, amlodipine and doxazosin;
anti-inflammatory agents such as glucocorticoids, betamethasone, dexamethasone, prednisolone, corticosterone, budesonide, estrogen, sulfasalazine, rosiglitazone, mycophenolic acid and mesalamine;
anti-neoplastic/anti-proliferative/anti-miotic agents such as paclitaxel, 5-fluorouracil, cisplatin, vinblastine, vincristine, epothilones, methotrexate, azathioprine, adriamycin and mutamycin; endostatin, angiostatin and thymidine kinase inhibitors, cladribine, taxol and its analogs or derivatives, paclitaxel as well as its derivatives, analogs or paclitaxel bound to proteins, e.g. Abraxane™;
anesthetic agents such as lidocaine, bupivacaine, and ropivacaine;
anti-coagulants such as D-Phe-Pro-Arg chloromethyl ketone, an RGD peptide-containing compound, heparin, antithrombin compounds, platelet receptor antagonists, anti-thrombin antibodies, anti-platelet receptor antibodies, aspirin (aspirin is also classified as an analgesic, antipyretic and anti-inflammatory drug), dipyridamole, protamine, hirudin, prostaglandin inhibitors, platelet inhibitors, antiplatelet agents such as trapidil or liprostin and tick antiplatelet peptides;
DNA demethylating drugs such as 5-azacytidine, which is also categorized as a RNA or DNA metabolite that inhibit cell growth and induce apoptosis in certain cancer cells;
vascular cell growth promoters such as growth factors, vascular endothelial growth factors (VEGF, all types including VEGF-2), growth factor receptors, transcriptional activators, and translational promoters;
vascular cell growth inhibitors such as anti-proliferative agents, growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin;
cholesterol-lowering agents, vasodilating agents, and agents which interfere with endogenous vasoactive mechanisms;
anti-oxidants, such as probucol;
antibiotic agents, such as penicillin, cefoxitin, oxacillin, tobranycin, daunomycin, mitocycin;
angiogenic substances, such as acidic and basic fibroblast growth factors, estrogen including estradiol (E2), estriol (E3) and 17-beta estradiol;
drugs for heart failure, such as digoxin, beta-blockers, angiotensin-converting enzyme (ACE) inhibitors including captopril and enalopril, statins and related compounds;
macrolides such as sirolimus (rapamycin) or everolimus; and
AGE-breakers including alagebrium chloride (ALT-711).

Other therapeutic agents include nitroglycerin, nitrous oxides, nitric oxides, antibiotics, aspirins, digitalis, estrogen, estradiol and glycosides. Preferred therapeutic agents include anti-proliferative drugs such as steroids, vitamins, and restenosis-inhibiting agents. Preferred restenosis-inhibiting agents include microtubule stabilizing agents such as Taxol®, paclitaxel (i.e., paclitaxel, paclitaxel analogs, or paclitaxel derivatives, and mixtures thereof). For example, derivatives suitable for use in the embodiments described herein include 2′-succinyl-taxol, 2′-succinyl-taxol triethanolamine, 2′-glutaryl-taxol, 2′-glutaryl-taxol triethanolamine salt, 2′-O-ester with N-(dimethylaminoethyl)glutamine, and 2′-O-ester with N-(dimethylaminoethyl)glutamide hydrochloride salt.

Other preferred therapeutic agents include tacrolimus; halofuginone; inhibitors of HSP90 heat shock proteins such as geldanamycin; microtubule stabilizing agents such as epothilone D; phosphodiesterase inhibitors such as cliostazole; Barkct inhibitors; phospholamban inhibitors; and Serca 2 gene/proteins. In yet another preferred embodiment, the therapeutic agent is an antibiotic such as erythromycin, amphotericin, rapamycin, adriamycin, etc.

In preferred embodiments, the therapeutic agent comprises daunomycin, mitocycin, dexamethasone, everolimus, tacrolimus, zotarolimus, heparin, aspirin, warfarin, ticlopidine, salsalate, diflunisal, ibuprofen, ketoprofen, nabumetone, prioxicam, naproxen, diclofenac, indomethacin, sulindac, tolmetin, etodolac, ketorolac, oxaprozin, celcoxib, alagebrium chloride or a combination thereof.

The therapeutic agents can be synthesized by methods well known to one skilled in the art. Alternatively, the therapeutic agents can be purchased from chemical and pharmaceutical companies.

### 5.2. Methods of Making the Medical Devices

In one method for making the medical devices described herein, the method comprises the step of providing a medical device having a substrate and at least one cavity disposed therein. The method further comprises disposing or forming a pellet in the cavity. Therapeutic agents can be disposed in at least some pores of the pellet.

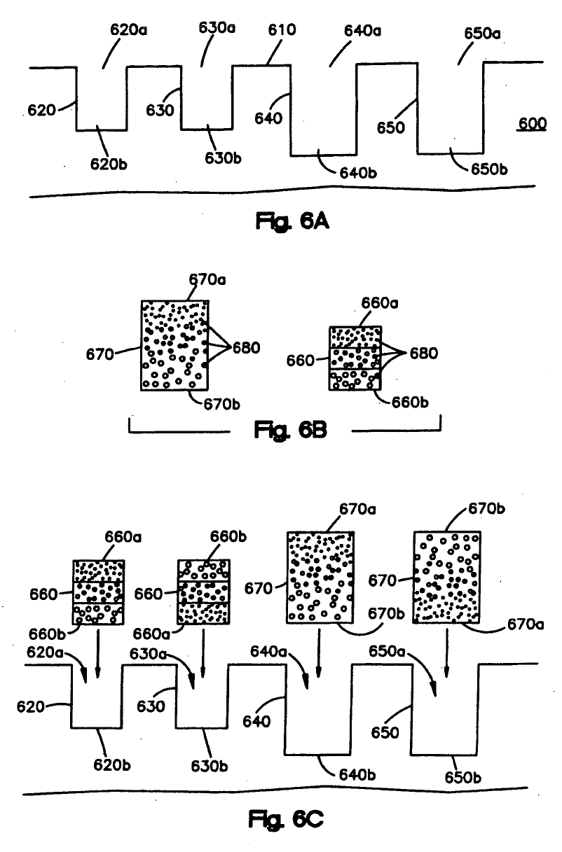

For instance, FIGS. 6A-6C show an example of a method for making the medical devices described herein. In this method, a medical device having a substrate 600, such as stent having a stent sidewall structure is provided (FIG. 6A). The substrate 600 has a surface 610 and at least one cavity disposed within the substrate 600. In this case, there are four cavities shown 620, 630, 640 and 650. Each of the cavities 620, 630, 640 and 650 has a first end 620a, 630a, 640a and 650a and a second end 620b, 630b, 640b and 650b opposing the first end. Each first end 620a, 630a, 640a and 650a comprises an opening that is in fluid communication with the stent sidewall structure surface 610 and each second end 620b, 630b, 640b and 650b comprises the bottom of a cavity.

As shown in FIG. 6B, pellets 660, 670 comprising a non-polymeric material having a plurality of pores 680 therein are formed. The pellets have a first end 660a and 670a and a second end 660b and 670b opposing the first end. Also, at least some of the pores have different pore sizes and the pores are arranged in a manner to form a pore size gradient in the pellet. Pellet 660 is made up of different layers each of which have varying pore sizes.

As shown in FIG. 6C, two pellets are disposed in cavities 620 and 640 in a manner so that first ends 660a and 670a face toward the openings 620a and 640a of cavities 620 and 640, respectively, and second ends 660b and 670b face toward the second ends 620b and 640b or bottoms of cavities 620 and 640, respectively. The two remaining pellets are disposed in the cavities 630 and 650 in a manner so that second ends 660b and 670b face toward the openings 630a and 650a of cavities 630 and 650, respectively, and first ends 660a and 670a face toward the second ends 630b and 650b or bottoms of cavities 630 and 650, respectively. A therapeutic agent can be disposed in at least some of the pores of the pellets before or after the pellets are disposed in the cavities. In alternative embodiments, the pores in the non-polymeric material can be formed after the material has been disposed in the cavity.

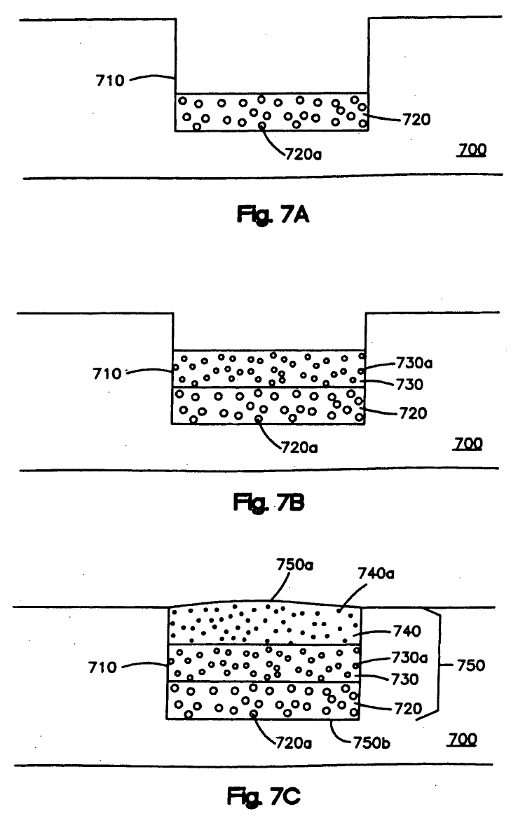

FIGS. 7A-C show another embodiment of a method for making the devices described herein. This method comprises the step of providing a medical device having a substrate 700 and at least one cavity 710 disposed therein. As shown in FIG. 7A, a pellet is formed in the cavity by disposing a first solid, non-polymeric material to form a first layer of the pellet 720. The first solid, non-polymeric material comprises a plurality of pores therein. At least some of the pores of the first layer 720 have a first pore size 720a. The pores can be formed in the first solid, non-polymeric material before or after it is disposed in the cavity 710.

Thereafter, as shown in FIG. 7B, a second solid, non-polymeric material is disposed over the first layer 720 to form a second layer 730 of the pellet. The second solid, non-polymeric material comprises a plurality of pores therein. At least some of the pores of the second layer have a second pore size 730a that is different from the first pore size 720a. The pores can be formed in the second solid, non-polymeric material before or after it is disposed in the cavity 710.

FIG. 7C shows that a third solid, non-polymeric material is disposed over the second layer 730 to form a third layer 740 of the pellet 750. The third solid, non-polymeric material comprises a plurality of pores therein. At least some of the pores of the third layer have a third pore size 740a that is different from the first and second pore sizes 720a, 730a. In this embodiment, the third pore size 740a is smaller than the second pore size 730a, which is smaller than the first pore size 720a. The pores can be formed in the third solid, non-polymeric material before or after it is disposed in the cavity 710. As shown in FIG. 7C, the pellet 750 comprises three layers 720, 730 and 740, and first and second opposing ends 750a and 750b. The first end of the pellet 750a faces toward the opening of the cavity and the second end of the pellet 750b faces toward the bottom of the cavity. A therapeutic agent can be disposed in at least some of the pores of the layers before or after the layers are formed in the cavity. Additionally, each layer can have the same or a different therapeutic agent.

5.2.1. Preparing Cavities in the Substrate

The cavities in the substrate can be created by any method known to one skilled in the art including, but not limited to, sintering, co-deposition, micro-roughing, laser ablation, drilling, chemical etching or a combination thereof. For example, the cavities can be made by a deposition process such as sputtering with adjustments to the deposition condition, by micro-roughening using reactive plasmas, by ion bombardment electrolyte etching, or a combination thereof. Other methods include, but are not limited to, alloy plating, physical vapor deposition, chemical vapor deposition, sintering, or a combination thereof. Still another suitable method that can be used to form the cavities involves the use of colloid crystals as templates to form porous materials. In such methods, colloid crystals are assembled to serve as a template. Voids between the crystals are filled with a material such as a sol-gel solution or suspension of metal nanoparticles. The material between the crystals is allowed to solidify and then the colloid crystals are removed. Examples of such processes are described in, O. Velev, et al., Colloidal crystals as templates for porous materials, Current Opinion in Colloid & Interface Sciences 5, 56-63, (2000), (hereinafter “Velev”) hereby incorporated by reference in its entirety.

Additionally, the cavities can be formed by removing a secondary material such as a spacer group from the material used to form the substrate. In particular, the substrate is formed from a composition containing the substrate material and the secondary material. The secondary material is then removed. Techniques for removing a secondary material include, but are not limited to, dealloying or anodization processes, or by baking or heating to remove the secondary material. The secondary material can be any material so long as it can be removed from the substrate material. For example, the secondary material can be more electrochemically active than the substrate material. Examples of a method for removing a secondary material are described in U.S. Publication No. 2005/0266040, which is incorporated by reference herein in its entirety.

5.2.2. Preparing The Pellets

The pellets described herein can be formed inside a cavity of a medical device substrate or, alternatively, the pellets can be formed prior to being disposed in a cavity of a medical device substrate. When forming pellets prior to disposing them in a cavity of a medical device substrate, the pellets described herein can be prepared by obtaining a non-polymeric material and shaping the material into pellets of desired sizes and shapes.

Other methods that can be used to form pellets include embossing techniques. An example of an embossing technique is described in C. Goh, et al., Nanostructuring Titania by Embossing with Polymer Molds Made from Anodic Alumina Templates, Nano Letters 5:8, 1545-1549 (2005), hereby incorporated by reference in its entirety. By using embossing techniques, large quantities of pellets can be formed using porous pellet molds made out of polymethyl methacrylate (PMMA). The molds can be used to emboss titanium oxide sol-gel solutions applied to a surface by spin coating. Once the sol-gel solution has dried the polymethyl methacrylate mold can be removed with acetonitrile. Also, the PMMA mold can be designed so that it can stamp out individual pellets.

Additionally, porous pellets can be formed in the cavities of the medical device substrate. In certain embodiments, individual porous layers that comprise a pellet can be each individually disposed in the cavity as shown in FIGS. 7A-7C. In the embodiments where the pellet comprises layers of material, the layers can be joined to each other by using an adhesive.

In other embodiments, sol-gel processes can be used to form porous pellets in the cavities of the medical device substrate. For example, sol-gel solutions containing polyethylene glycol (PEG) spacing elements can be disposed in a cavity. The PEG spacing elements can then be removed, leaving behind a porous pellet. Sol-gel solutions containing polyethylene glycol (PEG) spacing elements are discussed in B. Guo, et al., Sol gel derived photocatalytic porous TiO₂ thin films, Surface & Coatings Technology 198, 24-29 (2005) hereby incorporated by reference in its entirety. Layers of the sol-gel solutions comprising different molecular weight PEG spacing elements can be disposed in the cavities of the medical device substrate. The PEG spacing elements can then be removed, leaving behind a layered pellet having various sized pores in the pellet.

In certain embodiments of the methods described herein, pores are formed after the pellets have been formed and after the pellets have been disposed in the cavities. In alternative embodiments of the methods described herein, the pores in the pellets can be formed in the material used to make the pellets or the pores can be formed after the pellets are formed but prior to disposing the pellets in the cavities. The pores in the pellets or the material used to make the pellets can be formed using any of the techniques described above for making the cavities.

In embodiments where a therapeutic agent is disposed in pores, the therapeutic agent can be dispersed in the pores by any method known to one skilled in the art including, but not limited to, dipping, spray coating, spin coating, plasma deposition, condensation, electrochemically, electrostatically, evaporation, plasma vapor deposition, cathodic arc deposition, sputtering, ion implantation, use of a fluidized bed, or a combination thereof. Methods suitable for dispersing the therapeutic agent into the pores preferably do not alter or adversely impact the therapeutic properties of the therapeutic agent. In medical devices containing a plurality of pellets each pellet can include the same or a different therapeutic agent.

To facilitate the disposition of the therapeutic agent into the pores, the therapeutic agent can be placed into a solution or suspension containing a solvent or carrier. For instance, a solution containing the therapeutic agent can be formed and the pellet or non-polymeric material can be dipped into the solution to allow the therapeutic agent to be disposed in the pores. Furthermore, forming porous pellets that include a therapeutic agent prior to disposing the pellets in the cavities have many advantages. For example, the pellets can be formed and exposed to high temperatures without affecting the medical device. Additionally, disposing the drug in the pellets before disposing the pellets in the cavities of the medical device substrate prevents excess therapeutic agent from being disposed on the medical device substrate.

Once the pellets have been made, the pellets can be disposed in the cavities of the medical device substrate by, for example, piezo-driven positioning devices. A piezo-driven positioning device can be used to grip a pre-formed and in certain embodiments a drug-filled pellet, dispose the pellet in a cavity and using a gripper, squeeze the area around the cavity, for example a stent strut, in which the cavity is located on and secure the pellet in the cavity. Alternatively, an adhesive or other material can be used to affix the pellets in the cavities.

The description provided herein is not to be limited in scope by the specific embodiments described which are intended as single illustrations of individual aspects of certain embodiments. The methods, compositions and devices described herein can comprise any feature described herein either alone or in combination with any other feature(s) described herein. Indeed, various modifications, in addition to those shown and described herein, will become apparent to those skilled in the art from the foregoing description and accompanying drawings using no more than routine experimentation. Such modifications and equivalents are intended to fall within the scope of the appended claims.

All publications, patents and patent applications mentioned in this specification are herein incorporated by reference in their entirety into the specification to the same extent as if each individual publication, patent or patent application was specifically and individually indicated to be incorporated herein by reference. Citation or discussion of a reference herein shall not be construed as an admission that such is prior art.

What is claimed is:

1. An implantable stent comprising:
a. a stent sidewall structure having a surface;
b. at least one cavity, having first and second opposing ends, disposed within the stent sidewall structure wherein the first end of the cavity comprises an opening that is in fluid communication with the stent sidewall structure surface and the second end of the cavity comprises the bottom of the cavity;
c. at least one pellet disposed within the cavity comprising a non-polymeric material having a plurality of pores therein; and
d. a therapeutic agent disposed in at least some of the pores of the pellet.

2. The stent of claim 1, wherein the pellet has first and second opposing ends and wherein the first end of the pellet faces toward the first end of the cavity and the second end of the pellet faces toward the second end of the cavity.

3. The stent of claim 2, wherein at least some of the pores have different pore sizes.

4. The stent of claim 3, wherein the pores are arranged in a manner to form a pore size gradient in the pellet.

5. The stent of claim 4, wherein the pores having the largest pore size are disposed proximate the first end of the pellet.

6. The stent of claim 4, wherein the pore size gradient extends from the first end of the pellet to the second end of the pellet.

7. The stent of claim 2, wherein the pellet comprises one or more layers.

8. The stent of claim 7, wherein a first layer comprises pores having a first pore size and wherein a second layer comprises pores having a second pore size that is different from the first pore size.

9. The stent of claim 8, wherein the layers are arranged in a manner to form a pore size gradient in the pellet.

10. The stent of claim 9, wherein the pores having the largest pore size are disposed in the layer proximate the first end of the pellet.

11. The stent of claim 1, wherein the therapeutic agent comprises an anti-thrombogenic agent, anti-angiogenesis agent, anti-proliferative agent, antibiotic, anti-restenosis agent, growth factor, immunosuppressant or radiochemical.

12. The stent of claim 1, wherein the therapeutic agent comprises an agent that inhibits smooth muscle cell proliferation.

13. The stent of claim 1, wherein the therapeutic agent comprises paclitaxel.

14. The stent of claim 1, wherein the therapeutic agent comprises sirolimus, tacrolimus, pimecrolimus, everolimus or zotarolimus.

15. The stent of claim 1, wherein the stent sidewall structure surface is free of any coating.

16. An implantable intravascular stent comprising:
a. a stent sidewall structure comprising a plurality of struts each having an abluminal surface and a luminal surface,
b. at least one cavity, having first and second opposing ends, disposed within a strut, wherein the first end of the cavity comprises an opening that is in fluid communication with the abluminal surface of the strut and the second end of the cavity comprises the bottom of the cavity;
c. at least one pellet comprising a non-polymeric material, having a plurality of pores therein, disposed within the cavity; wherein the pellet has first and second opposing ends and wherein the first end of the pellet faces toward the first end of the cavity and the second end of the pellet faces toward the second end of the cavity; and wherein at least some of the pores have different pore sizes and the pores are arranged in a manner to form a pore size gradient in the pellet in which the pores having the largest pore size are disposed proximate the first end of the pellet; and
d. an anti-restenosis agent disposed within at least some of the pores of the pellet.

17. An implantable intravascular stent comprising:
a. a stent sidewall structure comprising a plurality of struts each having an abluminal surface and a luminal surface;
b. at least one cavity, having first and second opposing ends, disposed within a strut, wherein the first end of the cavity comprises an opening that is in fluid communication with the abluminal surface of the strut and the second end of the cavity comprises the bottom of the cavity;
c. at least one pellet comprising a non-polymeric material, having a plurality of pores therein, disposed within the cavity; wherein the pellet has first and second opposing ends and wherein the first end of the pellet faces toward the first end of the cavity and the second end of the pellet faces toward the second end of the cavity; and wherein the pellet comprises a first layer comprising pores having a first pore size, a second layer comprising pores having a second pore size that is smaller than the first pore size, and a third layer comprising pores having a third pore size that is smaller than the second pore size; and wherein the first, second and third layers are arranged in a manner to form a pore size gradient in the pellet in which the first layer is disposed proximate the first end of the pellet; and
d. an anti-restenosis agent disposed within at least some of the pores of the pellet.

18. A method for making an implantable stent comprising:
a. providing a stent having a stent sidewall structure having a surface and at least one cavity, having first and second opposing ends, disposed within the stent sidewall structure, wherein the first end of the cavity comprises an opening that is in fluid communication with the stent sidewall structure surface and the second end of the cavity comprises the bottom of the cavity;
b. disposing at least one pellet into the cavity, wherein the pellet comprises a non-polymeric material having a plurality of pores therein; and wherein the pellet has first and second opposing ends, and the first end of the pellet faces toward the first end of the cavity and the second end of the pellet faces toward the second end of the cavity; and wherein at least some of the pores have different pore sizes and the pores are arranged in a manner to form a pore size gradient in the pellet; and
c. disposing a therapeutic agent in at least some of the pores of the pellet.

19. The method of claim 18, wherein the pores having the largest pore size are disposed proximate the first end of the pellet.

20. The method of claim 18, wherein the pellet comprises one or more layers.

21. The method of claim 20, wherein a first layer comprises pores having a first pore size and wherein a second layer comprises pores have a second pore size that is different from the first pore size.

22. The method of claim 21, wherein the layers are arranged in a manner to form a pore size gradient in the pellet.

23. The method of claim 22, wherein the pores having the largest pore size are disposed in the layer proximate the first end of the pellet.

24. The method of claim 18, wherein the pores are formed in the pellet before the pellet is disposed in the cavity.

25. The method of claim 18, wherein the therapeutic agent is disposed in the pores before the pellet is disposed in the cavity.

26. A method for making an implantable stent comprising:
a. providing a stent having a stent sidewall structure having a surface and at least one cavity, having first and second opposing ends, disposed within the stent sidewall structure wherein the first end of the cavity comprises an opening that is in fluid communication with the stent sidewall structure surface and the second end of the cavity comprises the bottom of the cavity;
b. forming a pellet in the cavity, wherein the pellet has a plurality of layers and first and second opposing ends, in which the first end of the pellet faces toward the first end of the cavity and the second end of the pellet faces toward the second end of the cavity, comprising:
(1) disposing a first solid, non-polymeric material into the cavity to form a first layer of the pellet, wherein the first layer has a plurality of pores having a first pore size; and
(2) disposing a second solid, non-polymeric material into the cavity to form a second layer of the pellet disposed over the first layer, wherein the second layer has a plurality of pores having a second pore size; and
c. disposing a therapeutic agent in at least some of the pores of the first and second layers.

27. The method of claim 26, wherein the step of forming the pellet further comprises disposing a third solid, non-polymeric material into the cavity to form a third layer of the pellet over the second layer, wherein the third layer has a plurality of pores having a third pore size that is different from the first pore size and the second pore size.

28. The method of claim 27, wherein the layers are arranged in a manner to form a pore size gradient in the pellet.

29. The method of claim 27, wherein the third pore size is greater than the second pore size and the second pore size is greater than the first pore size.

30. The method of claim 26, wherein the therapeutic agent is disposed in the pores of the first, second or third layer before the layer is formed.
